(19) United States
(12) Patent Application Publication
(10) Pub. No.: US 2023/0395210 A1
(43) Pub. Date: Dec. 7, 2023

(54) A SYSTEM COMPRISING OF AN APPARATUS FOR MEASURING AND ACQUIRING DISTRESS DATA FROM THE HUMAN BODY

(71) Applicant: PERKANT TECH PRIVATE LIMITED, Indore (IN)

(72) Inventors: Lokant JAIN, Indore (IN); Saniya JESWANI, Indore (IN)

(73) Assignee: PERKANT TECH PRIVATE LIMITED, Indore (IN)

(21) Appl. No.: 18/033,887

(22) PCT Filed: Oct. 28, 2021
(86) PCT No.: PCT/IN2021/051033

§ 371 Date: May 5, 2023

(30) Foreign Application Priority Data
Oct. 28, 2020 (IN) 202021046907

(51) Int. Cl. G16H 10/60; A61B 5/00

(52) U.S. Cl. CPC G16H 10/60; A61B 5/0022

(57) ABSTRACT

A system comprising of an apparatus for measuring and acquiring distress data from the human body the user's finger is placed through the finger slot on the sensors and the data of body vitals is collected using the Photoplethysmogram (PPG) sensor on the PPG PCB and the temperature sensor on the temperature PCB. The Unique Identification Method (UIM) sensor is used to identify the user the data of body vitals and UIN is together transmitted to the server; there is a comparison of current data with the grouped analysed data of that UIN and a comparison of the current data with the grouped analysed data; if deviation is found the current data is flagged and stored and an alert is generated, if deviation is not found the current data is stored and added to grouped analysed data.

### FIELD OF INVENTION

The present invention relates to an apparatus for acquiring distress data from the human body. More particularly this distress can be psychological or physiological. The system measures and records vital signs of a user's body to generate a pattern. The system compares the recorded vital signs to the said pattern and checks for deviations. The system then identifies if there is a distress in the user's body and alerts the user about the same.

### BACKGROUND

Due to the pandemic of 2020 the general practice before entering any building premises, offices or a public place is to check a person's temperature by a no contact temperature sensor. The data is then noted by hand in a register and those who possess a higher than average body temperature are not allowed to enter the premises based on the above test alone.

In Indian Patent Application No. 202041030100, the invention is only able to detect Covid or other respiratory infections. The system mentions the use of infrared based thermal screening which is not accurate, and is not clinical grade. Only two body vitals are being recorded which are pulse and heart rate. The system also includes a lot of moving parts and hence might introduce artifacts in the measurement and records. The system is designed in a way that the user will have to be in contact with a lot of surfaces which can become a fomite in case of infectious diseases.

In Indian Patent 202011024968 the system uses sound waves for the detection of cough. This system is very unreliable as it requires a room with noise cancellation for higher accuracy. Also disclosed is the use of a contactless thermal scanner which are not meant for clinical use on humans and nor are they accurate enough. The system talks about detecting if a mask is present by using a webcam which causes a data security risk and is redundant against non-respiratory diseases. The system does not take body vital signs into consideration which are far more accurate with a wider scope of detection.

The Indian Patent 202041038022 uses commercially available basic microcontrollers which are costly; it also uses modules which makes the system more expensive. The system as disclosed in this invention is a combination of pre-existing apparatus. The temperature sensor used to take the skin temperature is primitive and not meant for human use, an accuracy of 1 degree c is obsolete; there is a need for a system which uses a clinical grade temperature sensor which has an accuracy of 0.1 degree c. This system only takes oxygen levels and pulse in consideration. There is a need for a system which can also measure and take respiration and blood pressure into consideration. The system requires WiFi for IoT(Internet of things) connectivity, there is a need for a system which uses a gsm network which has a wider network coverage and mobility, there is also a need for a system which can function locally to protect the data of the users. There is also a need for a system wherein the transmission is encrypted and safe, a system that uses machine to machine SIM cards. There is a need for a system which can be used for taking the data of hundreds of users instead of just one.

### OBJECT OF THE INVENTION

An object of current invention is to overcome all the sufferings of the prior art and provide a novel system.

Another object of the present invention is to log vital signs to create historical data or create a data pattern.

Another object of the present invention is to stop the formation of a cluster of communicable diseases by alerting the user about deviations in their data pattern.

Another object of present invention is to make the logged vital signs data available on any electronic media using the IoT(Internet of things) feature of the device.

Another object of present invention is to provide a novel and more accurate apparatus for screening users for infections before they enter the premises of a building or public space.

Another object of present invention is to provide a system which emphasises on the user's data security and uses only the prospective/retrospective systematic health records to sense patterns, the users personal data is never shared, the system is also capable of functioning with a local server.

Another object of present invention is to provide point of care devices/wearable devices to users, which are enabled with pattern recognition and use artificial intelligence.

Another object of present invention is to provide a solution for mass screening users for infection in the body.

Another object of present invention is to provide solutions for remote monitoring of vital signs of users and digitally sharing a log of historical data related to the user's vital signs.

### BRIEF DESCRIPTION OF DRAWINGS

FIG. 1 is a flow chart showing the working of the system with the following steps:

A user approaches the apparatus (1), the apparatus waits for the unique identification number (UIN) of the user (2). The UIN is verified by Radio Frequency Identification (RFID) (3) or a new users cell phone is used for creating a UIN (3A); user places a finger on the apparatus and the data of their body vitals is captured (4); data of body vitals and UIN is together transmitted to the analytics server (5); profiles corresponding to the respective UIN are searched (6); data of body vitals previously stored in the profile of the UIN is found (7); comparison of current data with the grouped analysed data of that UIN (8); comparison of the current data with the grouped analysed data (8A); filter for data into three broad grouped categories (9); deviation in calculated on the basis of comparison with grouped analysed data (10); deviation is checked (11); if deviation is found the current data is flagged and stored (12) an alert is generated and user may be stopped from entering the premises (13); if deviation is not found the current data is stored and added to grouped analysed data (12A) message generated and user may be allowed to enter premises (13A), analytics data can be securely accessed on local or webserver with UIN (14).

FIG. 2 is a digital diagram of the Cloud Server (200) and the Main Printed circuit board (PCB) (100) of the apparatus which consists of the antenna (111), power supply (112), modem (113). Photoplethysmogram (PPG) sensor (114), microcontroller (115), temperature sensor (116). Unique Identification Method (UIM) sensor (117) and display (118).

FIG. 3 is a 3D isometric view of the apparatus showing the antenna (111), display (118), finger slot (121) and casing (101).

FIG. 4 is a side sectional view of the apparatus showing the Photoplethysmogram (PPG) sensor (114) Temperature Sensor (116). Unique Identification Method (UIM) sensor (117). Finger Slot (121), display (118) and Casing (101)

FIG. 4a is a bottom view of the apparatus showing the Unique Identification Method (UIM) sensor (117), PPG PCB (119). Temperature PCB (120), and Casing (101)

FIG. 5 is a front sectional view of the apparatus showing the Unique Identification Method (UIM) sensor (117), antenna (111), casing (101) and Main PCB (100)

### SUMMARY OF THE INVENTION

The present invention relates to a system comprising of an apparatus for measuring and acquiring distress data from the human body which consists of at its one end a system on chip (100) comprising of (i) an antenna (111) (ii) a power supply (112) (iii) a modem (113) (iv) a Photoplethysmogram sensor (114) (v) a microcontroller (115) (vi) a temperature sensor (116) (vii) an Unique Identification Method sensor (117) and display (118) and at its other end a cloud server (200) which comprises of servers that receive information from the system on chip (100); the system on chip (100) identifies a user by the Unique Identification Method sensor (117) and calibrates the Photoplethysmogram sensor (114) and the temperature sensor (116) to remove the environmental artifacts and motion artifacts and sends only the calibrated information along with the Unique Identification Number [UIN] to the cloud server (200)

The present invention further relates to the cloud server (200) as mentioned above where the servers group a user of corresponding UIN along with other users of corresponding UIN by their physiological conditions

The present invention further relates to the cloud server (200) as mentioned above where the servers monitor and store physiological conditions of a user with a corresponding UIN by the information received from the system on chip (100) such that the servers detect deviations of the physiological conditions simultaneously from previously monitored and stored physiological conditions of the user of the corresponding UIN and of the group of users, the servers communicate the deviations to the system on chip (100) to generate either an alert or a message on the display (118)

The present invention also relates to a system comprising of an apparatus for measuring and acquiring distress data from the human body which consists of at its one end a system on chip (100) comprising of (i) an antenna (111) (ii) a power supply (112) (iii) a modem (113) (iv) a Photoplethysmogram sensor (114) (v) a microcontroller (115) (vi) a temperature sensor (116) (vii) an Unique Identification Method sensor (117) and display (118) and at its other end a local server that receives information from the system on chip (100) such that the system on chip (100) identifies a user by the Unique Identification Method sensor (117) and calibrates the Photoplethysmogram sensor (114) and the temperature sensor (116) to remove the environmental artifacts and motion artifacts and sends only the calibrated information along with the Unique Identification Number [UIN] to the local server.

The present invention also relates to a method comprising of an apparatus for measuring and acquiring distress data from the human body as mentioned above to perform the following steps:
a. A user approaches the apparatus
b. the apparatus waits for the unique identification number (UIN) of the user
c. the UIN is verified by Radio Frequency Identification (RFID) or a new users cell phone is used for creating a UIN
d. user places a finger on the apparatus and the data of their body vitals is captured
e. data of body vitals and UIN is together transmitted to the server
f. profiles corresponding to the respective UIN are searched
g. data of body vitals previously stored in the profile of the UIN is found
h. comparison of current data with the grouped analysed data of that UIN or comparison of the current data with the grouped analysed data
i. filter for data into three broad grouped categories
j. deviation in calculated on the basis of comparison with grouped analysed data
k. deviation is checked
l. if deviation is found the current data is flagged and stored or if deviation is not found the current data is stored and added to grouped analysed data
m. if deviation is found an alert is generated and user may be stopped from entering the premises or if deviation is not found a message generated and user may be allowed to enter premises
n. analytics data can be securely accessed on local or webserver with UIN

The present invention further relates to a method comprising of an apparatus for measuring and acquiring distress data from the human body as claimed in claim 4 to perform the following steps:
a. A user approaches the apparatus
b. the apparatus waits for the unique identification number (UIN) of the user
c. the UIN is verified by Radio Frequency Identification (RFID) or a new users cell phone is used for creating a UIN
d. user places a finger on the apparatus and the data of their body vitals is captured
e. data of body vitals and UIN is together transmitted to the server
f. profiles corresponding to the respective UIN are searched\
g. data of body vitals previously stored in the profile of the UIN is found
h. comparison of current data with the grouped analysed data of that UIN or comparison of the current data with the grouped analysed data
i. filter for data into three broad grouped categories
j. deviation in calculated on the basis of comparison with grouped analysed data
k. deviation is checked
l. if deviation is found the current data is flagged and stored or if deviation is not found the current data is stored and added to grouped analysed data
m. if deviation is found an alert is generated and user may be stopped from entering the premises or if deviation is not found a message generated and user may be allowed to enter premises
n. analytics data can be securely accessed on local or webserver with UIN

### DESCRIPTION OF THE INVENTION

The present invention will now be described with reference to the accompanying drawings wherein the same numerals are used to denote the same part. However, the drawings only illustrate the invention and in no way limit the invention.

The terms and words used in the following description and claims are not limited to the bibliographical meanings, but are merely used by the inventor to enable a clear and consistent understanding of the invention. The description given herein is only representative of the illustrative embodiments and examples. Only for the sake of convenience, the description has focused on a limited number of representative examples of the drawings. However, the drawings and/or illustrations and/or the appended claims in no way limit the scope of the invention. It is understood that modifications and/or alterations of the drawings/illustrations of this invention other than those specifically set forth herein may be achieved by those skilled in the art and that such modifications and alterations are to be considered as falling within the overall scope of this invention.

Terms used herein are used only in order to describe an exemplary embodiment rather than limiting the present invention. In this case, singular forms include plural forms unless interpreted otherwise in context.

The present invention relates to a system comprising an apparatus for measuring and acquiring distress data from the human body. More particularly this distress can be psychological or physiological. The apparatus is part of a system which measures and records vital signs of a user's body to generate a pattern. The system compares the recorded vital signs to the said pattern and checks for deviations. The system then identifies that there is a distress in the user's body and alerts the user about the same.

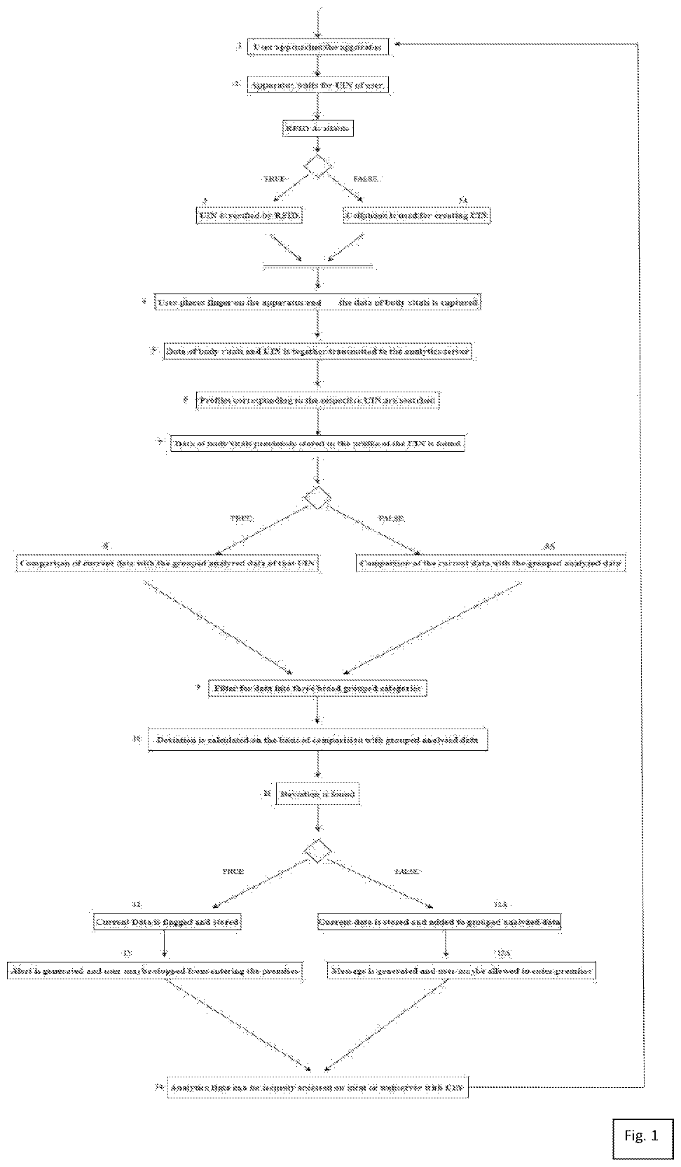

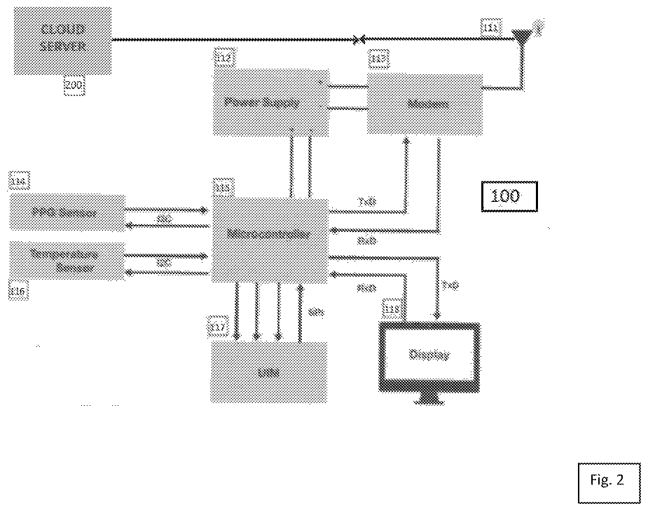

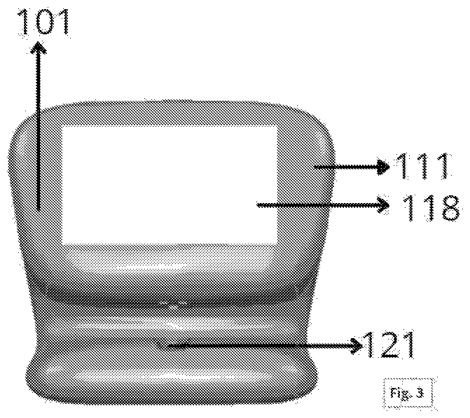

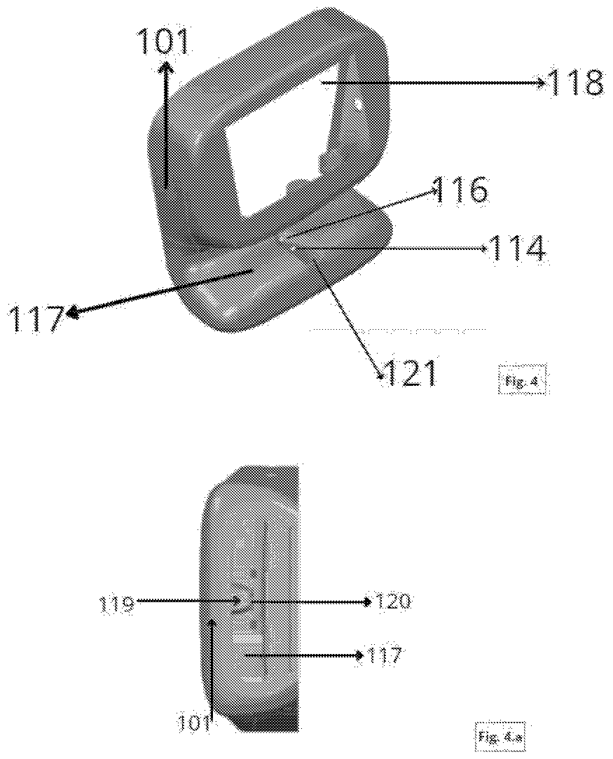

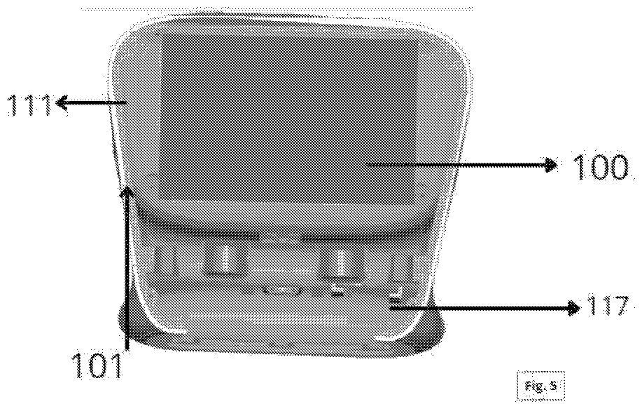

FIG. 1 shows a flow chart of the working of the system as described in the present invention, a user approaches the apparatus (1); the apparatus then scans for the unique identification number (UIN) of the user (2) which is preassigned to every user to identify them or assigned in step (3A). The UIN can be verified by Near Field Communication technology (NFC) of the mobile phone of the user or a RFID tag/card issued to the user (3) or a new user may scan a code from the display of the apparatus using their cell phone camera for creating their UIN (3A).

The purpose of a UIN is to log data to the user's profile on a local server using WiFi/Bluetooth or on a central analytics server in the cloud server (200) using IoT(Internet of things) for further analysis.

On successful identification the user places a finger (or wrist instead) on the apparatus. The apparatus captures the Photoplethysmogram (PPG) data and temperature data for a few seconds (4) together forming the data of body vitals.

This data of body vitals along with the UIN, apparatus serial number and ambient temperature is together transmitted (5) to the analytics server at the cloud server (200) using the installed modem, alternatively the system can even use Bluetooth and an electronic device with internet connectivity instead of a modem, alternatively the system can also function locally with a local server.

The analytics server then searches for the profile of the respective UIN (6), the data of body vitals previously analysed and stored in the profile of the UIN is found (7) the current data received is then compared with the ideal analysed data of the respective profile of the UIN (8), alternatively if the profile of the UIN is not found the analytics server identifies a group corresponding to the profile of the UIN and compares the data of body vitals received to the corresponding group of analysed data (8A).

The data is divided into three broad categories namely resting, active and transient (9), on the basis of grouped analysed data a comparison is made for the calculation of deviation (10).

The ideal threshold is determined by monitoring the person's routine generated parameters by scanning 2 or more times between 12 to 48 hours intervals.

The system can also use a machine learning model made on one dataset upon a completely different dataset due to the standardization and profiling.

If any deviations are found in the above comparison, the current data is flagged and stored to the respective profile of the UIN for analysis (12) an alert is sent (13) to the apparatus of the respective serial number and the respective user may be stopped from entering the premises. The alert may also be sent to the mobile app of the mobile device of the respective user.

If no deviation is found in the above comparison, the current data is stored and added to the respective profile of the UIN (12A) a message is sent to the respective apparatus (13) and the user may be allowed to enter the premises.

The system uses the ideal threshold values and the pattern of deviation created by them in subsequent scans to generate a prognosis of the specific type of distress.

When a UIN is newly created as mentioned in step (3A) the user enters their personal information that is their age, gender, blood group etc. The data of body vitals stored to the respective profile of the UIN as mentioned in steps (12) and (12A) is grouped by the analytics server at the could server (200) with respect to the aforesaid personal information; an artificial intelligence based pattern recognition algorithm analyses this grouped data to form groups of analysed data. The corresponding group of analysed data from the aforementioned groups is compared to the data of body vitals received at step (8A)

The data of body vitals is stored and added to the respective profile of the UIN as mentioned in steps (12) and (12A); the artificial intelligence based pattern recognition algorithm also separately analyses this data in each profile of the UIN to form the grouped analysed data of that profile.

For the calculation of this grouped analysed data the system does not require any prior health reports/disease/medicine prescription.

Grouped analysed data of each profile, along with previously recorded data of body vitals of each profile can be securely accessed on the web with the respective UIN upon verification.

FIG. 2 shows a digital diagram showing the apparatus connected to a cloud server (200) and PCB (100) the PCB (100) consists of the power supply (112) which powers the microcontroller (115) and the modem (113) the antenna (111) transmits information to the cloud server (200). The microcontroller (115) controls the remaining integrated circuits and makes sure the data is sent to the modem for transmission; the PPG sensor (114) and Temperature sensor (116) are controlled by the Microcontroller (115) the Unique Identification Method (117) identifies the user with the help of RFID of NFC signals by an ID card or other device given to the user, the Display (118) is used to provide instructions, messages and alerts to the users.

In step (1) of the steps described above and in FIG. 1, the user's finger is placed through the finger slot (121) on the sensors (114) and (116); the data of body vitals is collected using the Photoplethysmogram (PPG) sensor (114) on the PPG PCB (119) and the temperature sensor (116) on the temperature PCB (120) as shown in FIGS. 3 and 4 and 4a.

The Unique Identification Method (UIM) sensor (117) is used to identify the user on the basis of Unique Identification Number (UIN), or RFID or Bluetooth or biometrics etc. as mentioned in steps (3) and (3A) of FIG. 1.

The casing (101) encases all the components of the apparatus as shown in FIGS. 3, 4, 4a and 5

The modem (113) and antenna (111) are used to connect to the analytics server, database server and other servers at the cloud server (200) as shown in FIG. 2 or the local server/storage as mentioned in step (5), (13) and (13A) of the steps described above and in FIG. 1, a GPRS modem is used or a Bluetooth or Wifi device with internet connectivity can be used alternatively the modem can connect to a local server. The transmission over GSM is encrypted and safe due to the use of machine to machine SIM cards.

The system as disclosed in the present invention uses the steps as shown in FIG. 1 and apparatus as shown in FIG. 2 as a system for measuring and acquiring distress data in a human body. More particularly this distress can be psychological or physiological. The apparatus comprises a system which records vital signs of the body and generates a pattern which is analysed by the analytics server at the cloud server (200) and predictions may be made based on deviations in the said pattern. The system is capable of identifying distress without having any prior knowledge of health issues of the user in question.

The system uses Photoplethysmogram (PPG) sensor (114) to detect blood volume changes in the microvascular bed of tissue, using this data the system generates various vital signs of the body like Heart Rate, Blood Oxygen saturation (SPO₂) level. Respiratory Rate and Blood pressure which are displayed to the user through the display (118) and also uses other parameters such as Perfusion Index and Arterial Stiffness. The PPG sensor is automatically sanitised by a UV-C disinfection light fitted on the apparatus.

In another embodiment of the present invention the system also uses Electrocardiography/Electrocardiogram (ECG) sensors.

PPG uses an array of LEDs which illuminates the skin and measures changes in light absorption. This raw data is first passed through the inbuilt microcontroller (115) which uses a pattern-based process to clean the raw data against multiple parameters like skin colour, movement etc and generates a clean signal which is free of any artifact.

The system also captures body temperature with the help of a very precise medical grade temperature sensor (116) to record body temperature which is also displayed to the user through the display (18).

The apparatus is connected to a centralized cloud server/local analytics server and data logging capabilities are present using IOT(Internet of things).

The detection and prediction of physiological or psychological distress is done by the above said centralised cloud server/local server. The predictions/alerts are sent to the apparatus using above mentioned IoT (Internet of things) capabilities.

The present invention uses a custom designed circuit board containing antenna (111), power supply (112), modem (113), PPG sensor (114), microcontroller (115), temperature sensor (116). Unique Identification Method (UIM) sensor (117) as shown in FIG. 2. The present invention is a system comprising of a judicious combination of the aforementioned components and the steps as described in FIG. 1, with the microcontroller (115) on a custom made main PCB which synergistically generates a clean signal, free of any artifact. The aforementioned clean signal forms the ‘data of body vitals’ as mentioned above which is processed by the local/centralized analytics server at the cloud server of the present system. The system then generates multiple ‘grouped analysed data’ sets as per different attributes as mentioned above; any deviations from these sets of grouped data indicates a distress which may be physiological or psychological. Upon detection of distress the system alerts the user through a mobile app or through the display of the apparatus. The system also overcomes another challenge faced by the prior art which is that of high cost components used by hobbyist and students; the present invention has a custom-made main PCB which is cheaper and more advanced than devices using basic commercially available SOCs (System on a chip) and microcontrollers, the entire manufacturing process is done in India and designed in house making the apparatus more cost effective and portable.

### Examples of the Present Invention

While comparing the data (vitals) taken of the same user during a healthy/normal phase and when affected by an infectious disease such as coronavirus, several differences were observed. Even though initially most of these differences were minor, the present invention works efficiently to differentiate between the two situations.

Data collected by the apparatus of the present invention comprises of resting values, taken using the same equipment with clinical grade accuracy as described above. The user being screened is healthy, athletic and young (age 23, male). He has been regularly screened by the system and apparatus under normal conditions. Now, he contracts coronavirus and his vitals start deviating from the previous grouped analysed data of the subject, the system as described in the present invention recognises this deviation and alerts the aforementioned user.

For example, when the user was healthy his resting heart rate was 58 bpm but as soon as he got infected by the virus, his heart rate sped up to 85 bpm. His SPO2 levels didn't show much deviation but still dropped from a regular 100 to 98/99. Heart Rate Variability (HRV) was also affected and it increased from 1.47 to 3.78. Similarly, respiratory rate before and after contracting the virus was 12 and 14 respectively. The core body temperature wasn't affected much and just raised slightly from normal 98.5F to 99F. In case of blood pressure, human average is considered to be 120/80 mmhg but the average of the subject was 108/68 because he had hypotension-tendency towards low BP. The system and apparatus as described in the present invention during its regular use under healthy conditions recognised this behaviour and when the apparatus measured a 125/78 it alerted the user through the display of the apparatus, despite the values being in the accepted average range. This is because the values had a considerable deviation from the user's ‘ideal analysed data’ as described above.

The user in the above example was an asymptomatic carried of covid-19 with C-RP (C-reactive protein) baseline value of 0.8 (a healthy individual's C-RP ranges between 0-3.0). His clinical blood profile values and chest CT Scan suggested that he was healthy. The values being discussed in the previous paragraph are from the beginning of the onset of coronavirus infection, i.e. it had recently been contracted by the aforementioned user and there were no symptoms yet.

In this way with the use of personalisation and machine learning, the present invention is able to have an understanding of an individual's unique ‘ideal analysed data’ values and on the basis of this it is able to alert the user of any deviations. The system of the present invention is also smart enough to ignore variations in the data due to physical activities or weather conditions.

The raw data which was collected is also analysed against grouped analysed data of subjects with common attributes to infer more information like severity of the deviation and time of recovery etc, the inference is added to the grouped analysed data to further enrich the analysis.

Advantages of the apparatus described in the present invention:
i. The present invention can make reports of vital measurements easily accessible on any electronic media.
ii. The present invention can secure any premises from infected users.
iii. The present invention can provide systematic health records to develop predictive algorithms based on pattern recognition.
iv. The present invention can provide artificial intelligence enabled and pattern recognition point of care devices/wearable devices.
v. The present invention can predict distress or infection in the body without logging any prior health issues.
vi. The present invention can provide a solution for mass screening users for infection in the body.
vii. The present invention can aid in clinical decision-making.
viii. The present invention can make the household or untrained measurement of vital signs smart and more capable by logging data by using algorithm-based prediction which may aid medical practitioners when a user is in distress.
ix. The present invention can improve the efficiency of medical practitioners in Post Intervention/Post treatment/Postoperative monitoring of users.
x. The present invention can predict infection and distress even before the symptoms surface.
xi. The present invention can predict the rate of onset of infection in the body.
xii. The present invention can stop the formation of clusters for a communicable disease.

We claim:

1. A system comprising of an apparatus for measuring and acquiring distress data from the human body consisting of
at its one end a system on chip (100) comprising of (i) an antenna (111) (ii) a power supply (112) (iii) a modem (113) (iv) a Photoplethysmogram sensor (114) (v) a microcontroller (115) (vi) a temperature sensor (116) (vii) an Unique Identification Method sensor (117) and display (118)
and at its other end a cloud server (200) comprising of servers that receive information from the system on chip (100)
the system on chip (100) identifies a user by the Unique Identification Method sensor (117) and calibrates the Photoplethysmogram sensor (114) and the temperature sensor (116) to remove the environmental artifacts and motion artifacts and sends only the calibrated information along with the Unique Identification Number to the cloud server (200)

2. the cloud server (200) comprising of servers that receive information from the system on chip (100) as claimed in claim 1 wherein the servers group a user of corresponding UIN along with other users of corresponding UIN by their physiological conditions

3. the cloud server (200) comprising of servers that receive information from the system on chip (100) as claimed in claim 1 wherein the servers monitor and store physiological conditions of a user with a corresponding UIN by the information received from the system on chip (100)
the servers detect deviations of the physiological conditions simultaneously from previously monitored and stored physiological conditions of the user of the corresponding UIN and of the group of users
the servers communicate the deviations to the system on chip (100) to generate either an alert or a message on the display (118)

4. A system comprising of an apparatus for measuring and acquiring distress data from the human body consisting of
at its one end a system on chip (100) comprising of (i) an antenna (111) (ii) a power supply (112) (iii) a modem (113) (iv) a Photoplethysmogram sensor (114) (v) a microcontroller (115) (vi) a temperature sensor (116) (vii) an Unique Identification Method sensor (117) and display (118) and at its other end a local server that receives information from the system on chip (100) the system on chip (100) identifies a user by the Unique Identification Method sensor (117) and calibrates the Photoplethysmogram sensor (114) and the temperature sensor (116) to remove the environmental artifacts and motion artifacts and sends only the calibrated information along with the Unique Identification Method to the local server.

5. A method comprising of an apparatus for measuring and acquiring distress data from the human body as claimed in claim 1 to perform the following steps:
a. A user approaches the apparatus
b. the apparatus waits for the unique identification number (UIN) of the user
c. the UIN is verified by Radio Frequency Identification (RFID) or a new users cell phone is used for creating a UIN
d. user places a finger on the apparatus and the data of their body vitals is captured
e. data of body vitals and UIN is together transmitted to the server
f. profiles corresponding to the respective UIN are searched
g. data of body vitals previously stored in the profile of the UIN is found
h. comparison of current data with the grouped analysed data of that UIN or comparison of the current data with the grouped analysed data
i. filter for data into three broad grouped categories
j. deviation in calculated on the basis of comparison with grouped analysed data
k. deviation is checked
l. if deviation is found the current data is flagged and stored or if deviation is not found the current data is stored and added to grouped analysed data
m. if deviation is found an alert is generated and user may be stopped from entering the premises or if deviation is not found a message generated and user may be allowed to enter premises
n. analytics data can be securely accessed on local or webserver with UIN

6. A method comprising of an apparatus for measuring and acquiring distress data from the human body as claimed in claim 4 to perform the following steps:
a. A user approaches the apparatus
b. the apparatus waits for the unique identification number (UIN) of the user
c. the UIN is verified by Radio Frequency Identification (RFID) or a new users cell phone is used for creating a UIN
d. user places a finger on the apparatus and the data of their body vitals is captured
e. data of body vitals and UIN is together transmitted to the server
f. profiles corresponding to the respective UIN are searched\
g. data of body vitals previously stored in the profile of the UIN is found
h. comparison of current data with the grouped analysed data of that UIN or comparison of the current data with the grouped analysed data
i. filter for data into three broad grouped categories
j. deviation in calculated on the basis of comparison with grouped analysed data
k. deviation is checked
l. if deviation is found the current data is flagged and stored or if deviation is not found the current data is stored and added to grouped analysed data
m. if deviation is found an alert is generated and user may be stopped from entering the premises or if deviation is not found a message generated and user may be allowed to enter premises
n. analytics data can be securely accessed on local or webserver with UIN
